(19) United States
(12) Patent Application Publication
(10) Pub. No.: US 2004/0223940 A1
(43) Pub. Date: Nov. 11, 2004

(54) Method for removing the resin of the plant genus Rhus from skin or other surfaces

(75) Inventor: Jack Mentkow, Wellington, FL (US)

Correspondence Address: JACK MENTKOW, 1875 TULIP LANE, WELLINGTON, FL, 33414, US

(73) Assignees: Jack W. Mentkow; Lisa J. Mentkow

(21) Appl. No.: 10/153,572

(22) Filed: May 22, 2002

(51) Int. Cl. A61K 7/075; A61K 7/08; A61K 31/01

(52) U.S. Cl. 424/70.22; 424/70.24

(57) ABSTRACT

Resin of the genus Rhus is removed from the skin surface and/or subsurface as well as from other materials by application(s) of an effective amount of mineral spirits or related hydrocarbons alone or in combination with other ingredients acting as an allergen/resin imbibing/dissolving agent. A surfactant combined with or used subsequent to the mineral spirits or related hydrocarbons is applied to the mineral spirits treated area to facilitate removal by water or other liquid.

### REFERENCES CITED

U.S. patent documents

| | | | |
|---|---|---|---|
| 3862331 | January, 1975 | Crary | 424/331 |
| 4032662 | June, 1977 | Mannear | 514/675 |
| 4447418 | May, 1984 | Maddoux | 424/165 |
| 4451453 | May, 1984 | Lay et al. | 424/78.05 |
| 4879116 | November, 1989 | Fox et al. | 424/165 |
| 4895727 | January, 1990 | Allen | 424/642 |
| 5443847 | August, 1995 | West | 424/639 |
| 5888515 | March, 1999 | Albert et al. | 424/195 |
| 6113929 | September, 2000 | Karl | 424/401 |

### BACKGROUND OF THE INVENTION

The present invention is related to the use of a cleanser to extract and remove the plant resin/oil of plant species of the genus Rhus, the most common type of plant-induced dermatitis. All contain allergens consisting of a mixture of 3-substituted catechols. See Journal of the American Chemical Society, 76, 5070 (1954).

The allergens combine with epidermal proteins, forming complete antigens on conjugated proteins. Antibodies are synthesized by the endothelial system in response to the antigenic stimulus. The blood stream then transmits these antibodies to the cutaneous system where the antibodies affix themselves causing sensitization. See Handbook of Non-Prescription Drugs (pp. 172-175), published by the American Pharmaceutical Assoc. (1973).

### SUMMARY OF THE INVENTION

This invention relates to a method of removing the plant species resin/oil/allergen of the genus Rhus which comprises the tropical applications of an effective amount of mineral spirits which removes the resin/oil/allergen from the skin surface and underlying dermal tissue. By applying the mineral spirits and allowing the mineral spirits to remain in contact with the affected areas for a prescribed period of time, the allergen is effectively drawn to and taken up by the mineral spirits from the skin surface and underlying dermal tissue. This can then be removed from the skin surface by the use of a surfactant without spreading the allergen to other, unaffected, parts of the body.

It is critical that the mineral spirits be in contact with all parts of the skin exposed to the resin/oil/allergen and that the surfactant be thoroughly rinsed off with cool running water.

The applications of the mineral spirits are effective in preventing the outbreak of the rash after contact with plant species of the genus Rhus and are even effective in reducing the duration and intensity of the rash when applied after the rash has erupted. These, when followed or concurrent with the use of a surfactant which is then rinsed off with water.

### DESCRIPTION OF THE PREFERRED EMBODIMENTS

In accordance with the specification of the present invention, a two-step process is used to extract and remove the plant species of the genus Rhus resin/oil/allergen from affected areas of skin, underlying dermal tissue as well as other materials.

In the first step, a gel form of mineral spirits is applied liberally to the affected area of skin and is allowed to remain in contact with the skin for a minimum of two minutes to promote extraction from underlying dermal tissue in accordance with the following Table #1:

TABLE #1

| Ingredient | Amount (by weight) |
|---|---|
| Deodorized Mineral Spirits | 75% |
| Gelling Agent | 24.5% |
| Eucalyptus Oil | .5% |

The eucalyptus oil also has a soothing effect on the skin. The soap or surfactant suitably made from saponified fats containing one or more oil dissolving agents in the proportions indicated in the following Table #2 is applied to remove the now unlocked resin of the plant species of the genus Rhus which was imbibed by the mineral spirits and is now on the skin surface able to be rinsed off using cool running water.

TABLE #2

| Ingredient | Amount (by weight) |
|---|---|
| Glycerin Soap | 89.5% |
| Sodium Lauryl Sulfate | 10% |
| Oil of Eucalyptus | .5% |

The oil of eucalyptus also has a prolonged soothing effect on the skin. While the foregoing specifications describe a preferred embodiment of the present invention, one skilled in the art may make many modifications to the preferred embodiment without departing from the broader aspects of the invention. In particular, various substitutions can be made for deodorized mineral spirits as the resin imbibing hydrocarbon. In the surfactant, other ingredients can be used that are effective in dissolving surface oil.

To further illustrate the present invention, the following testing on various subjects which had been carried out over the last few years is presented below.

Case I

The subject was a young male who had been exposed to poison ivy a week earlier. At the time of treatment, the patient had open oozing sores on his buttocks, testicles, and down his thighs. The affected areas were treated with the invention. Within a few minutes, the subject stated that the itching had stopped. To ensure the removal of any residual oil/resin, another treatment was performed the following day and the sores dried within 24 hours.

Case II

The subject was an adult male previously determined to be extremely sensitive to urushiol, the resin/oil/allergen of the plant species of the genus Rhus. As a test, poison ivy was rubbed on his forearm. After one hour, with visible inflammation and significant itching already present, one arm was treated with the invention. The itching stopped immediately and the inflammation subsided within minutes.

Case III

The subject was an adult male previously determined to be extremely sensitive to urushiol. As a test, poison ivy was rubbed on his forearm. Over the next several days, the rash was allowed to erupt and sores to open and ooze. The subject was then treated with the invention. The subject stated that itching and inflammation subsided immediately. Treatment was repeated twice more and the sores had dried by the following day.

Case IV

The subject was a young female who had been exposed to poison ivy. At the time of treatment, the subject had a defined rash on one arm and one leg. The affected areas were treated with the invention. The subject stated that itching subsided immediately. The rash disappeared within 24 hours.

None of the subjects treated suffered any adverse side effects.

As illustrated by the aforementioned examples, the use of the invention is effective both before and after the rash erupts, even after the rash has blistered and begun oozing.

What is claimed is:

1. A method of removing/extracting plant resin from skin surface and subsurface and from other materials comprising the topical application of an effective amount of a resin imbibing hydrocarbon derivative followed by a surfactant to remove surface oils and resins.

2. The method of claim #1 wherein said hydrocarbon derivative is comprised of mineral spirits alone or in combination with other ingredients.

3. The method of claim #1 wherein the surfactant is comprised of a glycerin soap containing saponified fats and sodium lauryl sulfate whether combined with or separate from the mineral spirits.
